(19) United States
(12) Patent Application Publication
(10) Pub. No.: US 2010/0280477 A1
(43) Pub. Date: Nov. 4, 2010

(54) NAPPY

(76) Inventors: Rosalind Ann Henderson, Perthshire (GB); Melanie Jane Woods, Fife (GB)

Correspondence Address: Saddlers Cottage East, Main Street, Inchture, Perthshire, PH14 9RN, omitted

(21) Appl. No.: 12/600,465

(22) PCT Filed: May 19, 2008
(86) PCT No.: PCT/IB08/51962

§ 371 Date: Apr. 14, 2010

(30) Foreign Application Priority Data
May 17, 2007 (GB) 0709458.4

(51) Int. Cl. A61F 13/15

(52) U.S. Cl. 604/385.15; 604/386; 604/385.24

(57) ABSTRACT

A nappy comprising an outer cover (1) for wrapping around a wearer' s waist and bottom and for holding an inner absorbent layer (2) to the wearer, said outer cover (1) being securable about the wearer by fastening points (10,11) provided on front and rear areas thereof; wherein said outer cover (1) is foldable for adjusting its length such that, in use, the waist height of the nappy is adjusted; and wherein one of the front or rear areas of the outer cover (1) comprises a reversible panel (8), said reversible panel (8) having fastening points (11) provided on both sides thereof and being reversible for exposing fastening points (11) on an exterior surface of the outer cover (1) when said outer cover (1) is folded. In this way the size of the nappy can be adjusted to fit a particular wearer's waist height.

The present invention relates to a nappy/diaper, and in particular a two-part washable and reusable nappy system.

In this connection, with conventional nappies there is a choice between disposable or re-usable products. The re-usable nappies tend to fall into two main categories—‘All In One’ and ‘Two-Part’ systems. Both disposable and re-usable have a number of inherent problems.

Re-useable ‘All In One’ systems comprise an absorbent layer with integral waterproofing layer, and are of a similar design to a disposable nappy. After use, the nappy is washed and re-used. However, a problem exists in that the nappy takes a considerable time to dry after washing. This is because the waterproof layer reduces the rate of evaporation of liquid. In addition the ‘All in One’ system requires different sized nappies according to the size and weight of the wearer to ensure an optimal fit. As such, the purchase of several different products is required for the period from birth to toilet training of a baby/child, which is expensive.

Conventional re-useable ‘Two-Part’ systems have a shaped or flat absorbent layer, with separate waterproof wrap over the top. In cases where a shaped absorbent layer is used, this is generally made from T shaped absorbent fabric, where the longer length is positioned between the legs and shorter width wraps around the waist, overlapping and securing to the front section. However, such systems are generally bulky as the T shape of the absorbent layer and the waterproof wrap creates multiple layers of absorbent fabric and waterproof material around the baby's waist, where relatively little absorbency is needed. Furthermore, although in some cases the absorbent T shaped layer can be adjusted to fit a growing baby, different sizes of wrap are required as the baby grows. Moreover, changing a two-part nappy takes longer than an All In One or disposable nappy, as there are two or more layers to fit, one over another, in addition to corresponding fastening systems on both the absorbent layer and the wrap.

Another type of two part system is a flat nappy or ‘prefold’ nappy, which has a flat square of absorbent material that is folded to fit within an outer wrap. Problems identified with this system are that they can be especially difficult to fit with an active baby. This can also result in movement of the ‘prefold’ within the wrap and can cause discomfort and leakage. The different sizes of wrap required as the baby grows results in extra expense. Some ‘Two Part’ systems have a wrap with a ‘stuffable’ pocket into which a Flat nappy/diaper or ‘prefold’ can be placed then removed for laundering, however this system can be awkward to assemble, the ‘prefold’ is prone to movement within the pocket and can subsequently cause discomfort and leakage.

Disposable nappies are single use items that present significant ecological problems in terms of waste disposal, and the quantity of raw materials used—notably paper pulp, and crude oil in the production of plastic. Currently, disposable nappies, even bio-degradable products, do not bio-degrade readily in the landfill environment.

The present invention seeks to overcome or mitigate some of the problems associated with known nappies.

According to a first aspect of the present invention there is provided a nappy comprising an outer cover for wrapping around a wearer's waist and bottom and for holding an inner absorbent layer to the wearer, said outer cover being securable to the wearer by fastening points provided on front and rear areas thereof; wherein said outer cover is foldable for adjusting its length such that, in use, the waist height of the nappy is adjusted; and wherein one of the front or rear areas of the outer cover comprises a reversible panel, said reversible panel having fastening points provided on both sides thereof and being reversible for exposing fastening points on an exterior surface of the outer cover when said outer cover is folded.

In this way, the present invention allows the size of the nappy to be adjusted to fit a range of wearer/baby sizes, for example, a growing infant from newborn baby through to larger babies, including infants of nappy wearing age (i.e. birth to toilet-training). That is, the length of the outer cover can be shortened or lengthened whist still providing fastening points on the exterior of the nappy which can be used to secure the nappy. Furthermore, this is achieved without undue folding of the nappy, avoiding bulky gathering of fabric. Moreover, as the nappy can be used for a considerably extended lifetime, because its size is adjustable, the environmental impact compared to conventional nappies is reduced. As will be understood, the nappy could also be sized for adult wearers.

In a preferred embodiment, said reversible panel forms a pocket on the outer cover, said pocket being invertible to expose the fastening points of different sides of said reversible panel. In this way, by inverting the pocket, the fastening points can be presented on either the inner and outer surfaces of the outer layer as required, depending on which way the outer layer is folded.

Conveniently, when the nappy is in a shortened waist height configuration, said reversible panel is orientated over the inner surface of said outer cover and the outer cover is folded outwardly over itself to present the reversible panel on the exterior of the nappy. Correspondingly, when the nappy is in a lengthened waist height configuration, said reversible panel is orientated over the outer surface of said outer cover to present the reversible panel on the exterior of the nappy. In this way, in either configuration, the fastening points (which may be a fastening fabric such as velcro (RTM)) are presented on the front of the nappy for easy fastening and are not exposed on the interior of the nappy, which could otherwise cause irritation against the baby's skin.

Conveniently, the outer cover or the reversible panel includes a plurality of fastening points located around the waist circumference of the nappy for, in use, adjusting the waist circumference of the nappy. Conveniently, the plurality of fastening points located around the waist area are provided on the reversible panel. In this way, the waist size of the nappy can be adjusted to fit different sized babies.

Conveniently, the nappy further comprises the inner absorbent layer, said inner layer being removable from the outer cover. In this way, the inner absorbent layer can be removed or detached for disposal or, more preferably, for washing. Importantly, as the inner absorbent layer can be removed, it can be washed and dried separately, thereby allowing both the outer cover and the absorbent layer to be cleaned and dried ready for re-use quickly.

Preferably, the inner layer is longer than the outer cover in the direction of the length of the nappy, said inner layer being foldable to fit within the interior of the outer cover. In this way, the overhanging section of the inner layer can be folded into the interior of the nappy to provide additional absorbency in a particular area, for example the front of a baby boy.

In one embodiment, the other of the one of the front or rear areas of the outer cover comprises a further reversible panel.

In a preferred embodiment, the reversible panel is a front panel provided at or adjacent the front edge of the nappy, such that in use it is adjacent the region of the wearer's stomach. This enables the nappy to be easily secured when the baby is laying flat with their back down as a parent is changing them.

Conveniently, the outer cover comprises two ridges formed on the interior surface thereof which forms a channel for holding the inner layer in position. In this way, the inner layer is held in position during fitting and wear of the nappy, without having to be independently secured to the baby. This makes fitting of the nappy easier and makes the nappy more comfortable to wear. Furthermore, the ridges act to direct and hold solids and fluid in the absorbent layer.

Preferably, the two ridges are elasticated. In this way, a good fit is achieved between the ridges and the baby's bottom, thereby sealing the contents of the absorbent layer.

Conveniently, the channel has a depth substantially the same or greater than the thickness of the inner layer. In this way, the absorbent layer is held reliably in position.

Conveniently, the outer cover comprises a waterproof layer on the interior surface thereof. This permits easy cleaning of the outer layer component of the nappy and prevents solids or fluids from leaking into the outer material of outer cover.

Conveniently, the outer cover comprises elasticated elements, in use, for holding the sides of the outer cover to the wearer's legs.

Conveniently, the fastening points are shaped to form part of the decorative design of the nappy. In this way, the fastening points become part of the inherent fabric design.

According to a second aspect of the present invention there is provided a nappy cover for applying over an inner absorbent layer, said cover comprising: fastening means for securing the nappy cover around the wearer and for holding the inner absorbent layer to the wearer; and a plurality of ridges formed on the interior surface of said nappy cover, said ridges forming a channel for holding the inner absorbent layer in position.

In this way, the inner layer is held in position during fitting and wearing without the need for independently securing the inner layer to the wearer prior to the outer layer, which makes fitting easier and wearing more comfortable. Furthermore, the ridges act to direct and hold solids and fluid in the absorbent layer.

Examples of the present invention will now be described with reference to the accompanying drawings, in which:

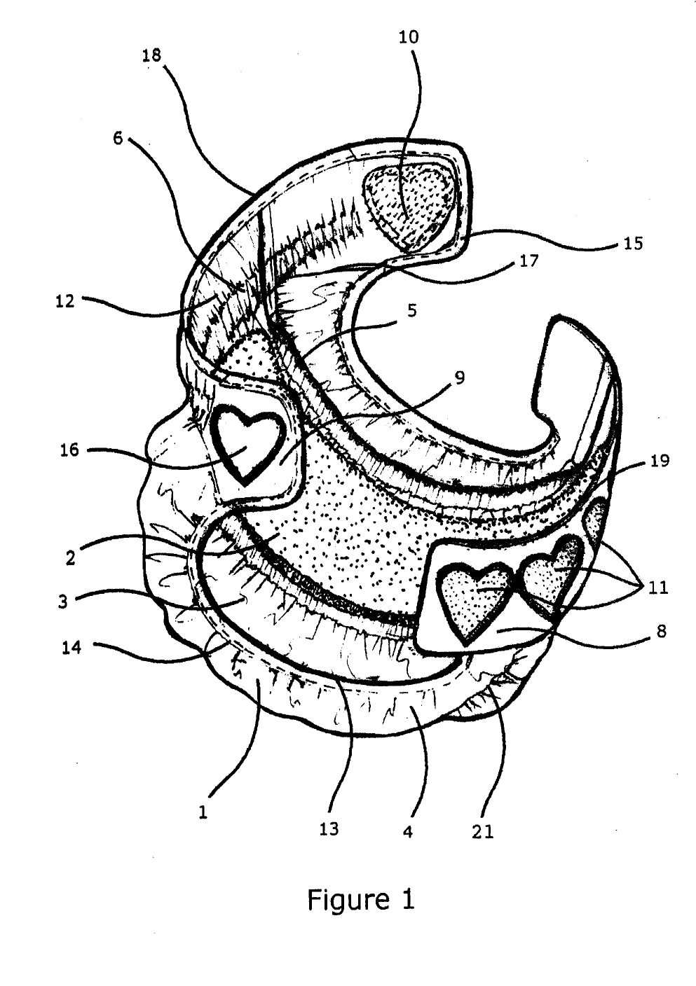

FIG. 1 shows a perspective view of a nappy according to a first embodiment of the present invention, showing the absorbent inner layer in position.

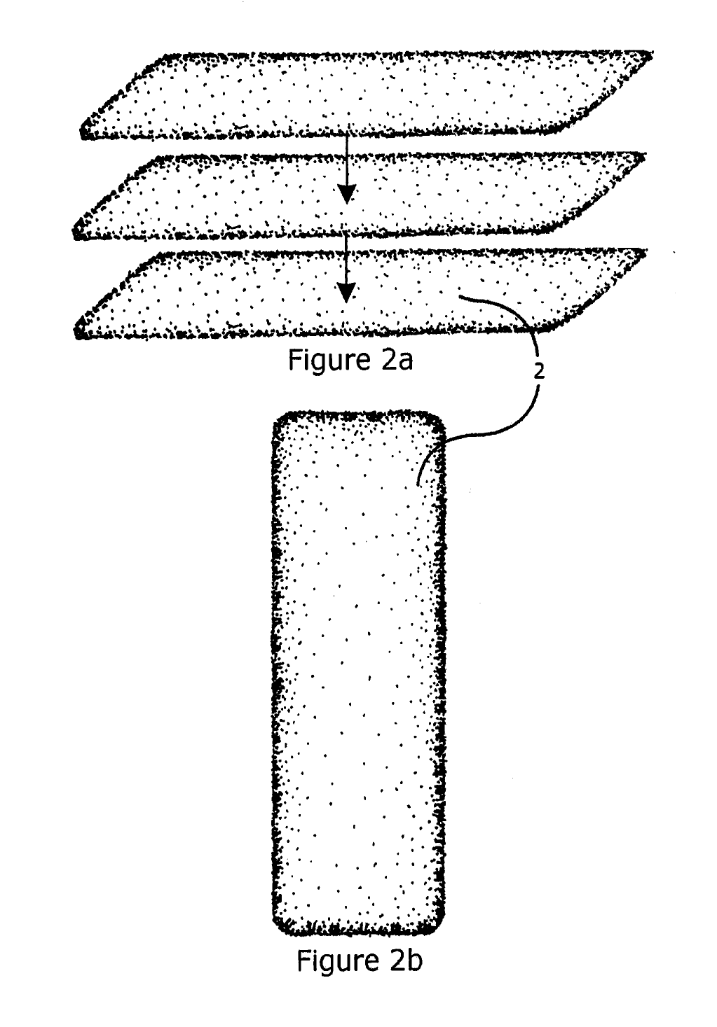

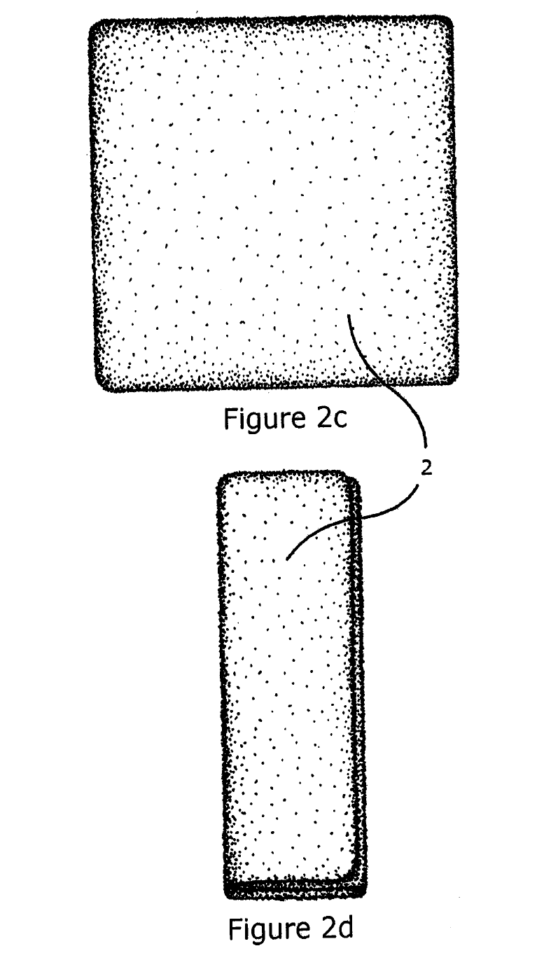

FIG. 2a shows an exploded view of the several layers of absorbent material forming of the absorbent inner layer.

FIG. 2b shows the absorbent inner layer shown FIG. 2a after the layers have been secured together.

FIG. 2c shows an alternative example of an absorbent inner layer.

FIG. 2d shows the absorbent inner layer of FIG. 2c in a folded position.

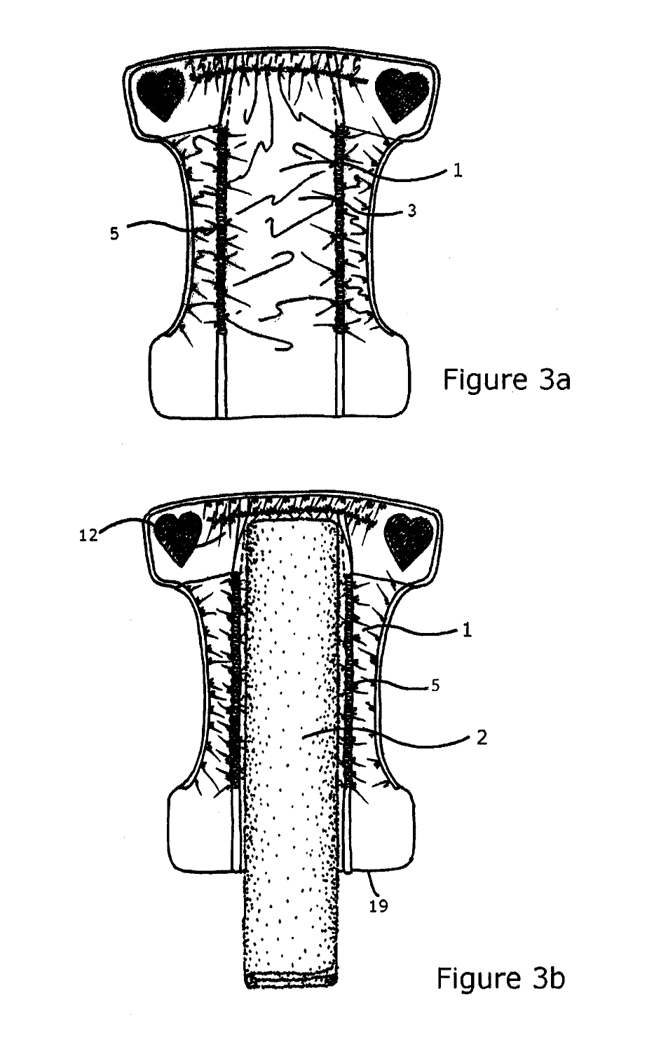

FIG. 3a shows a plan view of the inside of the waterproof outer wrap in a opened out position.

FIG. 3b shows a plan view of the inside of the waterproof outer wrap in a opened out position, with the absorbent inner layer present.

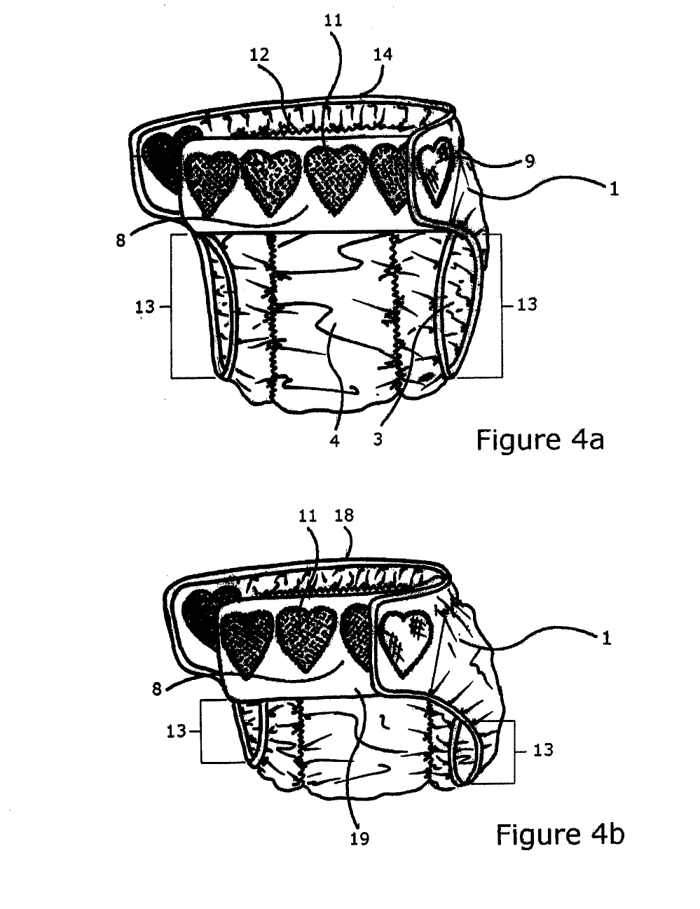

FIG. 4a shows a perspective view of the front of the waterproof outer wrap, where the front panel is folded to the extended position.

FIG. 4b shows a perspective view of the front of the waterproof outer wrap, where the front panel is folded to the retracted position.

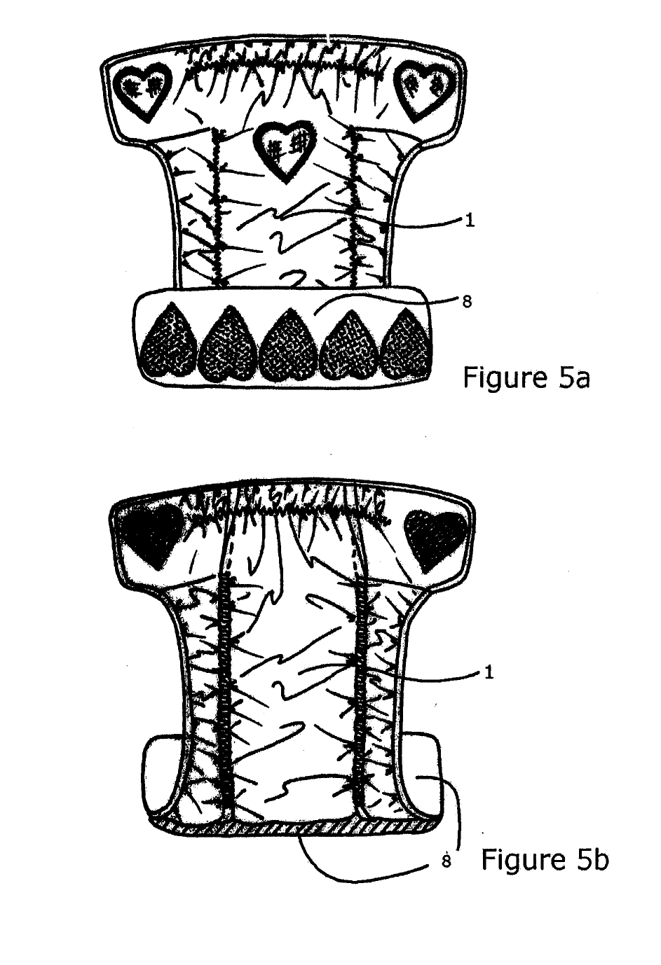

FIG. 5a shows a plan view of the outside of the waterproof outer wrap, where the front panel is folded to the retracted position.

FIG. 5b shows a plan view of the inside of the waterproof outer wrap, where the front panel is folded to the retracted position.

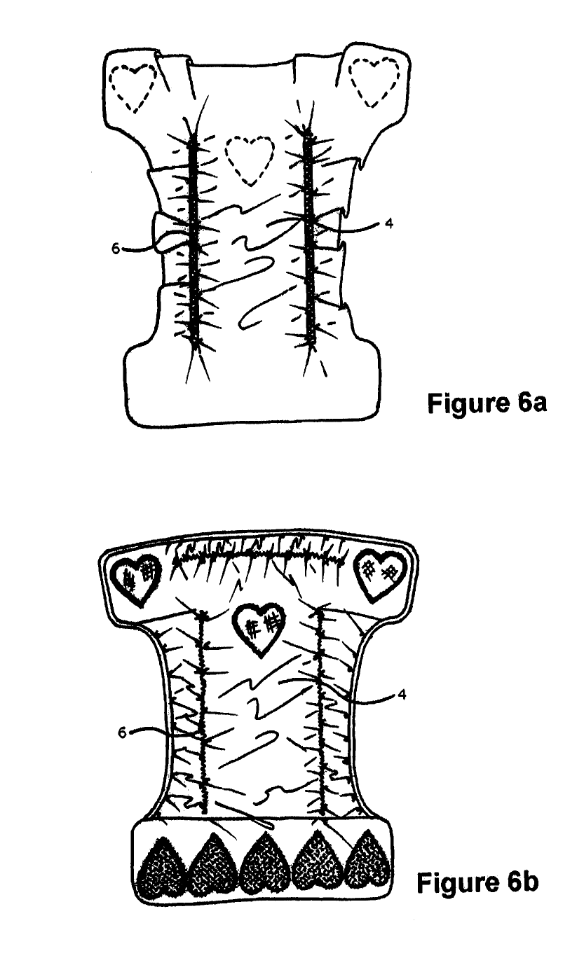

FIG. 6a shows a plan view of the inside of the outer material of the outer wrap.

FIG. 6b shows a plan view of the outside of the waterproof outer wrap.

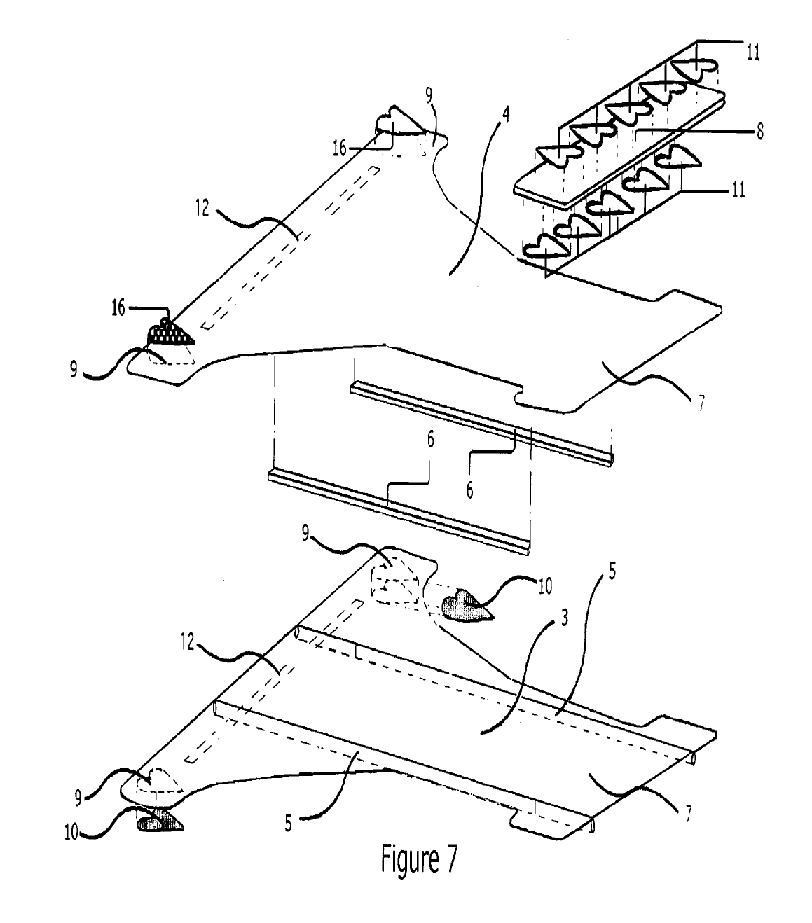

FIG. 7 shows an exploded view of the waterproof outer wrap.

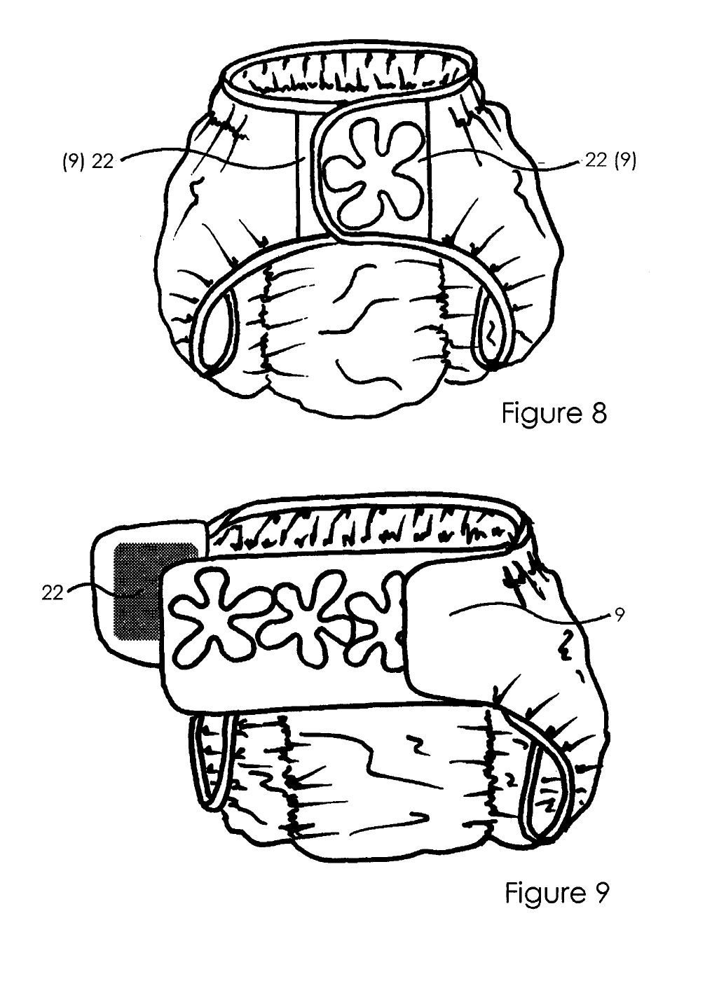

FIG. 8 shows a perspective view of the waterproof outer wrap in a reduced waist size position.

FIG. 9 shows a perspective view of the waterproof outer wrap in a enlarged waist size position

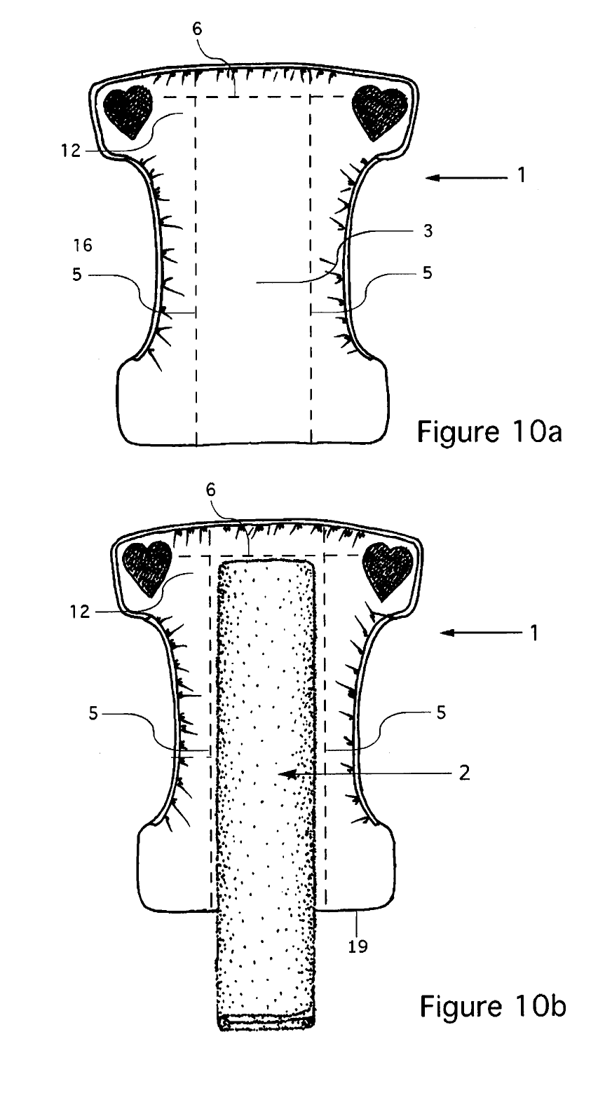

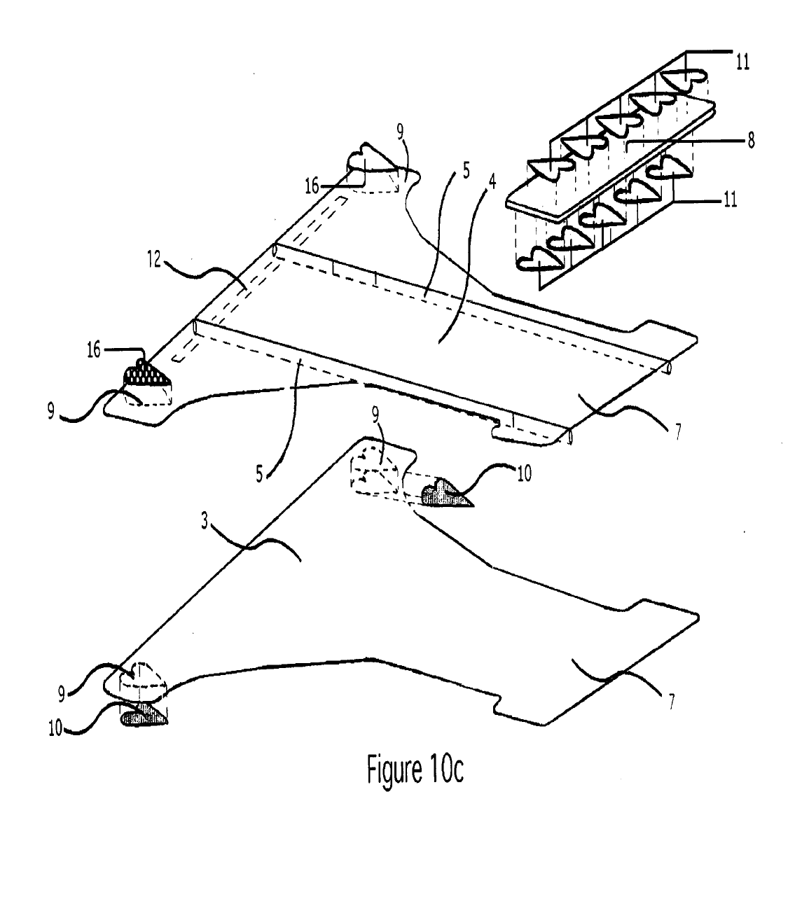

FIG. 10a shows a plan view of a second embodiment of the present invention.

FIG. 10b shows a plan view of the second embodiment shown in FIG. 10a, with the absorbent inner layer in position.

FIG. 10c shows an exploded view of the waterproof outer wrap of the second embodiment shown in FIG. 10a.

As described herein, the term “interior” is used to describe elements which, in use, face the inside of the nappy, i.e. towards the baby waring it. Conversely, the term “exterior” is used to describe elements which, in use, face the outside of the nappy, i.e. away from the baby wearing it. The term “inner” is generally used to denote elements which are nearer to the side of the nappy which, when the nappy is flat, would face a user (parent) about to fit the nappy, i.e. facing upward in FIG. 3a, for example. The term “outer” is generally used to denote elements which are nearer the side of the nappy which, when the nappy is flat, would face away from a user (parent) about to fit the nappy, i.e. facing downwardly in FIG. 3a, for example. It will also be understood that the term “baby” includes larger infants of a nappy wearing age.

FIG. 1 shows a first embodiment of the present invention. There is provided an absorbent inner layer 2 and a waterproof outer wrap or cover 1 which holds the inner layer against the baby. The absorbent inner layer 2 is held in place along the centre length of the outer wrap 1 by inner channel 5, which is formed by two elasticated ridges. The inner channel 5 thereby holds the absorbent inner layer 2 in position in the outer wrap 1 during fitting, but allows the absorbent inner layer 2 to be easily separated for washing and drying.

Adjustment to size of the nappy is made with the front panel 8, which is provided as a foldable/reversible double-sided panel, and a multi point fastening system 10&11. Furthermore, the incorporation of materials with elastic properties at the inner channel 5, the leg opening 13, rear waist-band 12, a stretchy gusset 21 ensure a good fit is achieved.

The outer wrap 1 is formed of an outer material 4 and an inner layer of waterproof/breathable fabric 3 suitable for next to the skin wear. The outer material 4 is formed of a light/medium weight fabric, permitting various colour-ways, patterns, textures and surface decorations to be used. This allows the visual design of the nappy to be easily modified. This is useful in that the surface decoration of the nappy is flexible.

The outer wrap 1 is shaped to fit around the wearer's legs and waist, with a stretchy rear area tapering to a crotch area and extending to a front area. Fastenings 10 and 11 are provided on the outer surface of both the front and rear areas such that, when fastened, both width and length of the outer wrap is secured. The fastenings 11 are provided at a number of discrete points around the front panel, allowing the waist size of the nappy to be easily adjusted, without unduly adding to the weight and bulk of the nappy.

As mentioned above, the rear waist-band 12 and leg opening 13 include elastic portions so that they are stretchy around their edges, which provides an adjustable and comfortable fit and prevents leakage of fluids and solids. Stretchy edging material 14, which in this example is provided by a folded-over elastic, secures the outer material and the inner waterproof/breathable fabric 3 together around the edges of each leg opening 13 and along the rear waistband edge 18. Edging material 15 is also secured around each fastening tab 9.

Further size adjustment of the waist-band 12 is achieved through the securing of elastic material 6 to the waterproof outer wrap 1 across the waist-band area.

A decorative motif 16 is provided , in this embodiment made from velcro (RTM), and forms part of the multi-point fastening system 10 and 11.

A dart 17 is provided at each side of the back of the outer material 4 and waterproof/breathable fabric 3. The dart 17 provides adjustment to the length of the leg opening and achieves a fuller back shape to help contain any solid/fluids and avoid leakage above the rear waist-band 12.

FIG. 2a shows the inner layer 2 which is formed of a number of layers of soft absorbent material, such as terry towelling material/fleece fabric. Absorbent inner nappy pads, formed of biodegradable and/or disposable and/or recyclable materials, could alternatively be used.

FIG. 2b shows the absorbent inner layer 2 once the layers have been secured together around its edges. The inner layer 2 has a width measurement compatible with the gap between the inner channel 5 of the waterproof/breathable fabric 3, as shown in FIG. 3b. Typically this width is approximately in the range of 9-13 cm in embodiments of the invention intended for a baby. In this specific example, the inner layer 2 is 11 cm wide.

FIG. 2c shows an alternative inner layer 2 example. The example is similar to the inner layer shown in FIGS. 2a and b, however it has a wider shape of absorbent fabric.

In this example, the inner layer 2 is folded in half, as shown in FIG. 2d, so that it is the same width as the inner layer shown in FIG. 2b, and thereby fits neatly between the inner channel 5, as shown in FIG. 3b. This has the advantage that the inner layer 2 can be folded out for washing and drying.

In a further alternative example, a traditional flat nappy may be used as the absorbent inner layer 2. This could be used, for example, for low birth weight babies and the flat nappy could be folded using a traditional method (e.g. kite fold) and would be held in place using a secure closing device (e.g. nappy nipper or safety pin). The waterproof outer wrap 1 could then be arranged into its smallest form, and fitted over the top of the traditional flat nappy in the same manner as shown in FIGS. 4b, 5a and 5b.

FIG. 3a shows a plan view of the inside of the waterproof outer wrap in a opened out position. The inner channel 5 is formed by folding the waterproof/breathable fabric 3 over two strips of elastic material and securing these to form two ridges. This creates two elasticated ridges, with the channel 5 there between. During fitting and wearing of the nappy, the inner channel 5 and its ridges form a barrier around the baby's leg that stretches for an adjustable fit and serve to hold the inner layer 2 in place, without the need for any other fastening device.

FIG. 3b shows the position of the absorbent inner layer 2 within the inner channel 5 of the waterproof outer wrap 1 ready for fitting to a wearer.

The absorbent inner layer 2 is shaped to fit within the area of the nappy that requires the most absorbency, i.e. within the crotch area. This limits the bulk of absorbent material needed, and so creates a slimmer fitting nappy. The ability to easily separate the absorbent inner layer 2 from the waterproof outer wrap 1 further allows the parts to be laundered separately and facilitates a shorter drying time after washing.

The length of the absorbent inner layer 2 is sufficient to fit from the rear waist-band 12 to the front edge 19 whilst stretched to fit the wearer. In the embodiment shown in FIG. 3b, there is sufficient length of inner layer 2 to fold the fabric to form a further layer at the front of the nappy. This provides extra absorbency at the front area, which is especially suitable for male wearer.

More than one, and/or a thicker absorbent inner layer 2 can be used to increase absorbency if required, eg. for night-time or for heavy wetters. Different types of materials or combinations of materials can be used, e.g. hemp/organic cotton for absorbent qualities; fleece to keep skin dry; silk to avoid irritation to delicate skin; or a biodegradable disposable/compostable liner.

The length of the outer wrap 1 can be adjusted to fit different size babies by adjusting the folding of the outer wrap 1 and front panel 8, which is attached in the front area 7 of the outer wrap 1 (see FIG. 7).

FIG. 4a shows a front view of the outer wrap 1 showing the exterior of the nappy. The front panel 8 is provided as a reversible panel forming a pocket having two sides (only the first being visible in this figure). FIG. 4a shows front panel 8 and outer wrap 1 is in its extended or lengthened configuration, so that it is folded out to the larger nappy size. This is shown in plan view in FIG. 6b.

The front panel 8 has a multi-point fastening fabric 11, such as hook and loop fabric or Velcro (RTM), attached on both sides thereof in a way that enables a corresponding fastening fabric 10 on the fastening tabs 9 to be secured. In this embodiment, for example, ‘female’ fastening fabric is provided on both sides of the front panel and ‘male’ fastening is provided on the fastening tabs 9.

When the outer wrap is folded to shorten its length from the position shown in FIG. 4a, the front area of outer wrap 1, including the front panel 8, is folded outwardly. In this way, the length of the nappy from the edge of the rear waist band 18 to the front edge 19 is reduced. In doing this, the first side of the front panel shown in FIG. 1 is folded towards the front of the outer material 4 so as to position the first fastening fabric pattern 11 next to this outer material. However, the reversible nature of the front panel 8 allows the front panel to be reversed such that the second side of the front panel 8 is folded out to the exterior of the nappy. That is, the reversible panel 8 can be inverted so as to be exposed on either the inner or outer surface of the outer wrap 1. Hence, when the outer wrap 1 is folded over to shorten it, which exposes its inner surface on the exterior of the nappy, the reversible panel is folded or inverted over this part of the inner surface so that it faces the exterior. In this way, the second pattern of ‘female’ fastening fabric 11 is presented on the exterior of the nappy to permit fastening. This is shown in FIG. 4b, and in plan view in FIGS. 5a and 5b. In this way, nappy is in a retracted or shortened configuration, with the waist length reduced. That is, the length from the bottom of the nappy to its top is reduced to fit smaller infants. At the same time, the leg circumference of leg opening 13 is also reduced.

This, combined with the stretch qualities of the waterproof outer wrap 1, creates a nappy that fits most infants of nappy wearing age (from birth to potty), and can be adjusted to fit any size of wearer. Moreover, the multi-point fastening 11 fabric can be formed into an attractive pattern forming part of the decorative design of the nappy.

Further adjustment to the width or waist circumference is achieved by a user (parent) pulling the fastening tabs 9 around the wearer's waist until the desired fit is achieved, and securing the ‘male’ fastening fabric 10 onto an area of the corresponding ‘female’ fastening fabric/device 11 on the front panel 8 or front panel area 7, as shown in FIG. 1. Tension in the elasticated rear waist-band 12 is used to ensure a good fit.

Adjustment of the rear waist-band 12 (as described above) will also affect and adjust the circumference and fit of the leg openings 13 around the wearers legs.

FIG. 5a shows the waterproof outer wrap 1 laid flat, with the front panel 8 folded to the larger size.

FIG. 5b shows the waterproof outer wrap 1 laid flat, with the front reversible double-side panel 8 folded to the smaller size.

FIG. 6a shows the inside of the outer material 4 of the outer wrap 1 before it is secured to the rest of the outer wrap 1. In order to retain the neat shape of the waterproof outer wrap 1 and facilitate an adjustable fit, elastic material (e.g. dress-makers elastic) is secured, while stretched, to the inside length of the outer material 4 to form a stretchy gusset 21 (see FIG. 1).

FIG. 6b shows the waterproof outer wrap 1 demonstrating the effect of the elastic material 6, described in FIG. 6a, creating a neat fitted shape to the outer material 4 that follows the shape of the waterproof/breathable fabric 3 created by the inner channel 5 (see also FIG. 3a) whilst retaining the stretch qualities needed to fit several sizes and shapes of wearer. For assembly details see also FIG. 7.

FIG. 7 is an exploded view of the waterproof outer wrap 1 (not to scale), showing that both the outer material 4 and the waterproof/breathable fabric 3 are cut to have an extended shape across the area which forms rear waist-band 12, and fastening tabs 9; and a shorter extended shape across the front panel area 7 to which the front reversible double-sided panel 8 can be attached and fitted with a fastening system (e.g. ‘female’ fastening fabric 11). The waterproof/breathable fabric 3 is wider than the outer fabric 4, until the ridges forming inner channel 5 are created along its length; thereafter making the outer material 4 and the waterproof/breathable fabric 3 silhouette the same. Both layers of fabric are attached only around the outer edge to avoid wicking of fluids through to the outer material 4.

FIGS. 8 and 9 shows an alternative embodiment of the present invention which is substantially identical to that described above. However, in this embodiment, a further ‘female’ fastening fabric is attached to the front of one (or both) fastening tabs 9. This allows the ‘male’ fastening fabric 10 on the inside of the fastening tab 9 to secure to it. This further ‘female’ fastening fabric is used in place of the decorative motif shown in FIG. 1. During fitting, the rear waist-band 12 is adjusted around the wearers waist until one fastening tab 9 crosses over the other at the front. It is then secured to the ‘female’ fastening fabric/device 11 on the front of the fastening tab 9 thereby allowing further size reduction (e.g. for very small babies).

FIG. 9 shows a further feature of the alternative embodiment shown in FIG. 8. As can be seen, fold-over tabs 22 are attached to the fastening tabs 9 forming a pocket. The fold-over tabs 22 are attached by the elastic edging material 15. The formed pocket can be inverted so that fold-over tabs 22 fold over fastening tabs 9, to thereby hide the male fastening fabric 10 provided on the inside of the fastening tab. This means that only fabric is exposed on the nappy, avoiding the fastening fabric sticking to other articles during washing or storage.

FIG. 10a shows a further alternative embodiment of the present invention in which elasticated channel 5 is incorporated in the outer material 4 of the outer wrap 1, on the inside of the material. Both the elasticated channel 5 and elastic material 6, secured across the rear waistband 12, are concealed between waterproof/breathable fabric 3 and the outer material 4 (see FIG. 10c). That is, once the outer material has been secured to inner waterproof material, the elastic material 6 is sandwiched between the two layers of the outer wrap and so the material itself is concealed from sight after manufacture. In this way, the elasticated material 6 forms ridges on the interior surface of the waterproof/breathable fabric 3. This has the effect of creating a neat fitted shape whilst retaining the stretch qualities needed to fit several sizes and shapes of wearer, and keeping the waterproof/breathable fabric 3 free from securing devices other than around the edges (i.e. reducing puncture of the fabric by stitching or other means) thus avoiding compromising the waterproof qualities of the fabric, and being easier to clean by avoiding folding and gathering. The positioning of the inner layer 2 is shown in FIG. 10b.

Although the present invention has been described in the above illustrated embodiments, the present invention is not limited solely to these particular embodiments and various modifications could be envisaged by the skilled person that fall within the scope of the present invention.

For example, the present invention allows for the option of using a decoratively shaped multi point fastening system 10 and 11, (e.g. hook and loop fabric) on the front panel 8 or front panel area 7 and fastening tabs 9 or fold-over tabs 22. These are both decorative and functional, and may include decorative top-stitching or edge-stitching or printing, or be combined with other decorative elements. Where a hook and loop system or other fastening material is used, the hook ‘male’ fastening fabric/device 10, is preferably secured onto the inside of the each fastening tab 9 and can be shaped to correspond to the decorative elements of the ‘female’ fastening fabric/device 11. The design of the multi point fastening system 10 and 11 is not limited to the designs shown in the illustrations, but can be of many alternative shapes and patterns.

Other fastening systems could also be used instead of hook and loop fabric (e.g. poppers, press catches, ties or gripping tapes), whereby a decorative element could be retained across the front panel area 7 or front reversible double-sided panel 8, (e.g. through an applied design or print) on to which the fastening system would be secured at appropriate intervals. Corresponding fasteners would also replace the hook ‘male’ fastening fabric/device 10 on the fastening tabs 9.

As an alternative to the above described examples, length of the outer wrap 1 could be adjusted by way of a back reversible double-sided panel that is attached to the rear waist-band 12 and fastening tabs 9 of the waterproof outer wrap 1, instead of, or as well as, the front reversible double-sided panel 8. In one such embodiment the ‘male’ fastening fabric/device 10 is secured to the insides of each fastening tab 9 and to the reverse side of the back reversible double-sided panel 20.

Furthermore, the present invention may also be provided with a fastening to retain the folded outer wrap and revisable panel in position when it is folded into a shortened configuration. Such a fastening could be provided on the outer material of the outer wrap for securing two of its layers together when they are folded over. The fastening could also form part of the decorative design of the nappy.

With the present invention, as the outer material is formed of mostly light to medium weight fabric, many colour-ways, prints, applique, texture can be easily incorporated into the design without compromising the effectiveness of the product. This forms an important role in the fashion direction and visual appeal of the product.

Furthermore, it will be understood that although in the above examples have been described in reference to a baby or toddler, the present invention is equally applicable to adult nappies.

Finally, as will be appreciated from the above, the present invention permits ecological issues to be addressed by allowing natural materials to be incorporated into the design. For example, by using the superior natural absorbency and anti-bacterial qualities of hemp; the softness, absorbency and durability of organic cotton; and by using organically grown, recycled, locally produced materials and processes where possible. Furthermore, as the nappy is washable, re-usable, functions from birth to toilet-training, it provides an extended life cycle and allows for use by more than one or subsequent children. As such, less raw materials are used in production, transport and packaging, compared to conventional re-usable and, more significantly, disposable nappies/diapers.

1. A nappy system comprising:
a washable and reusable outer wrap for wrapping around a wearer's waist and bottom and for holding an inner absorbent nappy to the wearer, said outer wrap being securable around the wearer by fastening points provided on front and rear areas thereof; wherein said outer wrap is foldable for adjusting its length such that, in use, the waist height of the nappy is adjusted; and
wherein one of the front or rear areas of the outer wrap comprises a reversible panel, said reversible panel having fastening points provided on both sides thereof and being reversible for exposing fastening points on an exterior surface of the outer wrap when said outer wrap is folded.

2. A nappy according to claim 1 wherein said reversible panel forms a pocket on the outer wrap, said pocket being invertible to expose the fastening points of different sides of said reversible panel.

3. A nappy according to claim 1 or 2 wherein, when the outer wrap is in a shortened waist height configuration, said reversible panel is orientated over the inner surface of said outer wrap and then the outer wrap is folded outwardly over itself to present the reversible panel on the exterior of the outer wrap.

4. An outer wrap according to any preceding claim wherein, when the outer wrap is in a lengthened waist height configuration, said reversible panel is orientated over the outer surface of said outer cover to present the reversible panel on the exterior of the outer wrap.

5. An outer wrap according to any preceding claim wherein the outer cover of the reversible panel includes a plurality of fastening points located around the waist circumference of the outer cover for, in use, adjusting the waist circumference of the nappy.

6. An outer cover according to claim 5 wherein the plurality of fastening points located around the waist area are provided on the reversible panel.

7. A nappy system according to any preceding claim, further comprising the inner absorbent nappy, said inner nappy being removable from the outer cover.

8. A nappy according to claim 7 wherein the inner nappy is longer than the outer cover in the direction of the length of the nappy system, said inner nappy being foldable to fit within the interior of the outer cover.

9. A nappy system according to any preceding claim wherein the other of the one of the front or rear areas of the outer cover comprises a further reversible panel.

10. An outer wrap according to any preceding claim, wherein the reversible panel is a front panel provided at the front edge of the nappy, such that in use it is adjacent the region of the wearer's abdomen.

11. An outer wrap according to any preceding claim wherein the outer cover comprises two ridges formed beneath the interior surface thereof, said ridges forming a channel for holding the inner nappy in position.

12. An outer wrap according to claim 11 wherein the two ridges are elasticated.

13. A nappy according to claim 11 or 12 wherein the channel has a depth substantially the same or greater than the thickness of the inner nappy.

14. A nappy system according to any preceding claim wherein the outer cover comprises a waterproof layer on the interior surface thereof.

15. A nappy system according to any preceding claim wherein the outer cover comprises elasticated elements, in use, for holding the sides of the outer cover to the wearer's legs.

16. A nappy system according to any preceding claim wherein the fastening points are shaped to form part of the decorative design of the nappy system, without unduly adding to the weight and bulk

17. An outer wrap for applying over an inner absorbent nappy, said outer wrap comprising:
fastening means for securing the outer wrap around the wearer and for holding the inner absorbent nappy to the wearer; and
a plurality of ridges formed beneath the interior surface of said outer wrap, said ridges forming a channel for holding the inner absorbent nappy in position.

18.

19. An outer wrap substantially as hereinbefore described with reference to the accompanying drawings.
